(19) United States
(12) Patent Application Publication
(10) Pub. No.: US 2017/0360882 A1
(43) Pub. Date: Dec. 21, 2017

(54) COMPOSITION FOR PREVENTING, IMPROVING OR TREATING PSORIASIS COMPRISING IMMUNOMODULATOR AND GLUCOSAMINE

(71) Applicant: DONGGUK UNIVERSITY INDUSTRY-ACADEMY COOPERATION FOUNDATION, Seoul (KR)

(72) Inventor: Ai-Young Lee, Seoul (KR)

(21) Appl. No.: 15/533,657

(22) PCT Filed: Sep. 25, 2015
(86) PCT No.: PCT/KR2015/010162

§ 371 Date: Jun. 7, 2017

(30) Foreign Application Priority Data
Dec. 9, 2014 (KR) 1020140175935

(51) Int. Cl. A61K 38/13; A61K 31/7008

(52) U.S. Cl. CPC A61K 38/13; A61K 31/7008

(57) ABSTRACT

The present invention relates to a composition for preventing, improving or treating psoriasis containing an immunomodulator and glucosamine. Cyclosporine, an immunomodulator, is difficult to administer for a long time due to side effects thereof, and, when the administration of cyclosporine is discontinued, lesions tend to deteriorate to an original state thereof. However, according to the present invention, the co-administration of cyclosporine and glucosamine, even when the dose of cyclosporine is reduced, may exert a far superior effect on the treatment or improvement of psoriasis than the administration of cyclosporine and glucosamine alone.

### TECHNICAL FIELD

The present invention relates to a composition for preventing, improving or treating psoriasis containing immunomodulator and glucosamine.

### BACKGROUND ART

Psoriasis is an immune-mediated autoimmune skin disease induced by chronic inflammatory cell infiltration in the skin and dysregulation of epidermal keratinocytes. Histologically, psoriasis is characterized by epidermal thickening and scaling, and hyperproliferative parakeratosis in skin biopsies. The mechanism by which psoriasis is induced has not yet been fully elucidated, and has been reported to be related to interactions between inflammatory cytokines and immune cell infiltration. A recent study demonstrated that T helper 17 (Th17) cells and Th17-mediated cytokines, such as IL-17A, IL-22 and IL-23, induced psoriasis.

Common treatments for psoriasis include topical and internal medications and phototherapy. In topical treatments, steroids, coal tar, anthralin, vitamin D3 and analogs thereof, retinoids, sunlight, and the like are used. These topical treatments have side effects of skin thinning, stretch marks, burns, irritation, and photosensitivity. In addition, steroids may induce tolerance and affect subsequent steroid therapies. Phototherapy involves the administration of psoralen in combination with ultraviolet B or A, which may promote skin aging and increase skin cancer incidence.

In addition, the most severe psoriasis is treated with immunomodulators, such as cyclosporine, which is currently the most commonly used medication. However, careful observation is needed because nephrotoxicity or hypertension may occur upon long-term use of cyclosporine. Therefore, to administer cyclosporine, approved as a therapeutic agent for long-term treatment of psoriasis, it is important to develop treatment methods capable of effectively treating psoriasis while reducing the administration dose. Thus, studies on such methods have been made, but there are no known effective treatment methods yet.

### DISCLOSURE

### Technical Problem

The present invention was made to solve the above problem. The present inventors developed a method of effectively treating psoriasis. As a result, it was confirmed that co-administration of cyclosporine and glucosamine remarkably improved the effectiveness of anti-psoriasis treatment while reducing the administration dose of cyclosporine. Based on these findings, the present invention was completed.

Thus, the present invention is directed to providing a pharmaceutical composition for preventing or treating psoriasis and a cosmetic composition for improving psoriasis, containing cyclosporine and glucosamine as active ingredients.

However, the technical problem to be solved by the present invention is not limited to the above-mentioned problem, and other problems not mentioned herein can be clearly understood by those skilled in the art from the following description.

### Technical Solution

One aspect of the present invention provides a pharmaceutical composition for preventing or treating psoriasis and a cosmetic composition for improving psoriasis, containing cyclosporine and glucosamine as active ingredients.

In one embodiment of the present invention, the cyclosporine and glucosamine may be contained in a weight ratio of 1:5 to 1:2000.

In another embodiment of the present invention, the cyclosporine may be cyclosporine A.

Another aspect of the present invention provides a pharmaceutical composition for enhancing the effectiveness of anti-psoriasis treatment, which is used in combination with an anti-psoriasis agent, wherein the pharmaceutical composition contains glucosamine and the anti-psoriasis agent is cyclosporine.

In one embodiment of the invention, the composition and the anti-psoriasis agent may be administered simultaneously, separately or sequentially.

Still another aspect of the present invention provides a method of treating psoriasis, the method including a step of co-administering cyclosporine and glucosamine to an individual.

Yet another aspect of the present invention provides a method of enhancing the effectiveness of anti-psoriasis treatment, the method including a step of co-administering cyclosporine and glucosamine to an individual.

### Advantageous Effects

Cyclosporine, an immunomodulator, is difficult to administer for a long time due to side effects thereof, and when the administration of cyclosporine is discontinued, lesions tend to deteriorate to an original state thereof. However, according to the present invention, the co-administration of cyclosporine and glucosamine, even when the dose of cyclosporine is reduced, can exert a far superior effect on the treatment or improvement of psoriasis than the administration of cyclosporine and glucosamine alone.

### DESCRIPTION OF DRAWINGS

FIG. 1 illustrates a result of measuring several representative symptoms using cumulative scores (erythema+scaling+thickening) after administering glucosamine (100, 200, 300 and 400 mg/kg) alone to an IMQ-induced psoriasis-like mouse model.

FIG. 2 illustrates a result of measuring several representative symptoms using cumulative scores (erythema+scaling+thickening) after administering cyclosporine A (10, 20 and 40 mg/kg) alone to an IMQ-induced psoriasis-like mouse model.

FIG. 3a illustrates a result of measuring a clinical skin score in each case after co-administering low doses of glucosamine (300 mg/kg) and cyclosporine A (10 and 20 mg/kg) to an IMQ-induced psoriasis-like mouse model.

FIG. 3b includes images showing the clinical characteristics of mice in each case after co-administering low doses of glucosamine (300 mg/kg) and cyclosporine A (10 and 20 mg/kg) to an IMQ-induced psoriasis-like mouse model.

FIG. 4a illustrates a result of measuring IL-17A levels in blood serum samples after co-administering low doses of glucosamine (300 mg/kg) and cyclosporine A (10 and 20 mg/kg) to an IMQ-induced psoriasis-like mouse model.

FIG. 4b illustrates a result of measuring IL-17A levels in skin samples after co-administering low doses of glucosamine (300 mg/kg) and cyclosporine A (10 and 20 mg/kg) to an IMQ-induced psoriasis-like mouse model.

FIG. 5a illustrates a result of measuring IL-22 levels in blood serum samples after co-administering low doses of glucosamine (300 mg/kg) and cyclosporine A (10 and 20 mg/kg) to an IMQ-induced psoriasis-like mouse model.

FIG. 5b illustrates a result of measuring IL-22 levels in skin samples after co-administering low doses of glucosamine (300 mg/kg) and cyclosporine A (10 and 20 mg/kg) to an IMQ-induced psoriasis-like mouse model.

FIG. 6a illustrates a result of measuring IL-23 levels in blood serum samples after co-administering low doses of glucosamine (300 mg/kg) and cyclosporine A (10 and 20 mg/kg) to an IMQ-induced psoriasis-like mouse model.

FIG. 6b illustrates a result of measuring IL-23 levels in skin samples after co-administering low doses of glucosamine (300 mg/kg) and cyclosporine A (10 and 20 mg/kg) to an IMQ-induced psoriasis-like mouse model.

FIG. 7a illustrates a result of measuring TNF-α levels in blood serum samples after co-administering low doses of glucosamine (300 mg/kg) and cyclosporine A (10 and 20 mg/kg) to an IMQ-induced psoriasis-like mouse model.

FIG. 7b illustrates a result of measuring TNF-α levels in skin samples after co-administering low doses of glucosamine (300 mg/kg) and cyclosporine A (10 and 20 mg/kg) to an IMQ-induced psoriasis-like mouse model.

FIG. 8 includes images representing H&E staining for back skin samples after co-administering low doses of glucosamine (300 mg/kg) and cyclosporine A (20 mg/kg) to an IMQ-induced psoriasis-like mouse model.

FIG. 9 illustrates a result of measuring the epidermal thickness of back skin samples after co-administering low doses of glucosamine (300 mg/kg) and cyclosporine A (20 mg/kg) to an IMQ-induced psoriasis-like mouse model.

FIG. 10 illustrates a result of measuring spleen size after co-administering low doses of glucosamine (300 mg/kg) and cyclosporine A (20 mg/kg) to an IMQ-induced psoriasis-like mouse model.

FIG. 11 illustrates a result of measuring spleen weights after co-administering low doses of glucosamine (300 mg/kg) and cyclosporine A (20 mg/kg) to an IMQ-induced psoriasis-like mouse model.

FIG. 12 illustrates a result of measuring the number of CD4⁺ CD25⁺ Treg cells after co-administering low doses of glucosamine (300 mg/kg) and cyclosporine A (20 mg/kg) to an IMQ-induced psoriasis-like mouse model.

### MODES OF THE INVENTION

Hereinafter, the present invention is described in detail.

The present invention provides a composition for preventing, improving or treating psoriasis containing cyclosporine and glucosamine as active ingredients, the composition including a pharmaceutical composition and a cosmetic composition.

According to the present invention, when cyclosporine, approved as a therapeutic agent for psoriasis, is administered in combination with glucosamine, the effectiveness of anti-psoriasis treatment is remarkably improved while reducing the administration dose of cyclosporine, thereby reducing the side effects of cyclosporine.

In the present invention, “cyclosporine” is an immunomodulator which suppresses in vivo immune responses, and cyclosporine A is preferably used, without being limited thereto.

In the present invention, “glucosamine” is produced through decomposition of chitosan, one of the main components of saltwater crab and shrimp exoskeletons. The main components of saltwater crab and shrimp exoskeletons are chitin and chitosan. Chitin is composed of 2-acetylamino-2-deoxy-β-D-glucose (N-acetylglucosamine), and chitosan is poly (β-(1,4)-glucosamine), a polysaccharide produced by deacetylation of chitin. Glucosamine may be represented by Formula 1 below.

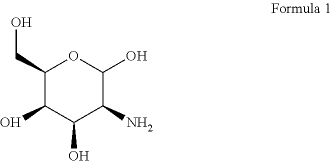 [SMILES: N[C@@H]1C(O)O[C@H](CO)[C@H](O)[C@@H]1O]

In the present invention, glucosamine derivatives may be used instead of glucosamine. “Glucosamine derivatives” may be represented by Formula 2 below, wherein the hydrogen atoms of the hydroxyl groups of glucosamine may be substituted with acyl or alkyl compounds.

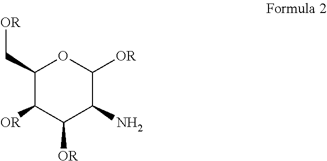

In Formula 2, R is an acyl group having 2 to 18 carbon atoms or a straight or branched chain alkyl group having 1 to 5 carbon atoms, and is preferably an acyl group, such as an acetyl propionyl, butyryl, pentanoyl, hexanoyl, heptanoyl, octanoyl, nonanoyl, decanoyl, undecanoyl, lauryl, tridecanoyl, myristyl, pentadecanoyl, palmitoyl, margaryl or stearyl group, or an alkyl group, such as a methyl, ethyl, propyl, butyl, pentyl, isopropyl, isobutyl or sec-butyl group.

In addition, glucosamine or glucosamine derivatives used in the present invention may be contained in the pharmaceutical composition of the present invention in the form of a pharmaceutically acceptable salt depending on the use of the pharmaceutical composition. For example, the salt form includes sulfates of glucosamine or glucosamine derivatives, hydrochlorides of glucosamine or glucosamine derivatives, maleates of glucosamine or glucosamine derivatives, and the like, without being limited thereto. A preferred salt form is a sulfate of glucosamine or glucosamine derivatives.

In the composition according to the present invention, cyclosporine and glucosamine are preferably contained in a weight ratio of 1:5 to 1:2000, more preferably 1:5 to 1:500, and most preferably 1:5 to 100.

The composition according to the present invention may be formulated into oral preparations, such as powders, granules, tablets, capsules, suspensions, emulsions, syrups and aerosols, and sterile injectable solutions, suppositories and preparations for percutaneous administration according to conventional methods. Examples of carriers, excipients and diluents that may be contained in the composition may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate and mineral oil. The composition may be formulated using diluents or excipients, such as fillers, extenders, binders, wetting agents, disintegrants and surfactants, when the diluents or excipients are required.

The composition according to the present invention is preferably formulated into a solid preparation for oral administration. The solid preparation for oral administration may include tablets, pills, powders, granules, capsules, and the like. The solid preparation may be formulated by mixing one or more excipients, for example, starch, calcium carbonate, sucrose or lactose, gelatin, and the like, as active ingredients. In addition to excipients, lubricants such as magnesium stearate and talc may also be used.

In addition, the composition according to the present invention is preferably formulated into a liquid preparation for oral administration. The liquid preparation for oral administration may include suspensions, liquids for internal use, emulsions, syrups, and the like. In addition to commonly used inert diluents, for example, purified water, ethanol and liquid paraffin, the liquid preparation may include various excipients, for example, wetting agents, sweeteners, flavoring agents, preservatives, and the like.

In addition, the composition according to the present invention may be formulated into a preparation for parenteral administration. The preparation for parenteral administration may include sterilized aqueous solutions, non-aqueous solvents, suspensions, emulsions, freeze-dried preparations, and suppositories. As the sterilized aqueous solutions, suitable buffers such as Hank's solution and Ringer's solution or physically buffered saline may be used. As non-aqueous solvents and suspensions, vegetable oils such as propylene glycol, polyethylene glycol and olive oil and injectable esters such as ethyl oleate may be used. When required, preservatives, stabilizers, wetting agents or emulsifying agents, and salts and/or buffers for controlling osmotic pressure may be used. In the case of suppositories, Witepsol, macrogol, Tween 61, cacao butter, laurinum, glycerogelatin, and the like may be used as common base compounds of suppositories.

When the composition of the present invention is administered to patients, the total daily usage of the composition may be determined by prescription within the scope of medical judgment. To determine a therapeutically effective dose for a particular patient, various factors including whether other agents are used, age, body weight, general health status, sex, diet, administration time, administration route, secretion rate of a composition and treatment period and similar factors well known in the medical field are preferably taken into consideration.

Preferably, cyclosporine is co-administered with glucosamine in an amount of 0.025 to 2.9 mg/kg, and the number of doses may be appropriately adjusted depending on a patient's skin condition and may be from one to three times per day. In addition, cyclosporine may be taken in combination with glucosamine up to a maximum dose of 5 mg/kg depending on the condition of skin lesions.

In addition, the composition according to the present invention may be formulated into an external preparation for application to the skin, a cosmetic preparation or the like. The external preparation for application to the skin may include ointments, plasters, sprays, suspensions, emulsions, creams, gels, and the like.

In addition to the above-described active ingredients, the cosmetic composition according to the present invention, when required, may be compounded with various ingredients (e.g., water-soluble components, powder components, oils, surfactants, moisturizers, viscosity controlling agents, preservatives, antioxidants, perfumes, pigments, and the like) generally used in external preparations for application to the skin, provided that the effect of the composition of the present invention is not deteriorated.

The formulation of the cosmetic composition of the present invention may be arbitrarily selected, and may be formulated into conventional forms such as skin ointments, essences, whitening creams, lotions, emulsions, packs, general lotions, skin milks, creams, serums, cosmetic soaps, softening lotions, medicated lotions, hair tonics, body cleansers, and oil gels. The cosmetic composition of the present invention may additionally contain one or more additives selected from the group consisting of oils, water, surfactants, moisturizers, thickeners, chelating agents, pigments, preservatives and perfumes, and is preferably formulated into softening lotions, nutritional lotions, nutritional creams, massage creams, nutritional serums, essences, and packs.

By applying the cosmetic composition of the present invention to a skin inflammation site, psoriasis may be alleviated and improved. The amount of application may be appropriately adjusted depending on the inflammation site of a patient, and the rate of application may be one to five times per day.

As another aspect of the present invention, the present invention provides a pharmaceutical composition for enhancing the effectiveness of anti-psoriasis treatment containing glucosamine, which is used in combination with cyclosporine, an anti-psoriasis agent. The composition and the anti-psoriasis agent may be administered simultaneously, separately or sequentially.

Hereinafter, preferred examples of the present invention are described to facilitate understanding of the present invention. However, the following examples are provided only for the purpose of easier understanding of the present invention, and the present invention is not limited by the following examples.

### EXAMPLES

### Example 1. Experimental Preparation and Experimental Methods

### 1-1. Preparation of Experimental Animals

8-week-old female C57BL/6 mice were purchased from Orient Bio (Seongnam, Korea). Mice were acclimated for a week before performing experiments and maintained in experimental animal facilities at the Dongguk University School of Medicine. All studies were approved by the Animal Care and Use Committee of Dongguk University Hospital.

### 1-2. Preparation of Drugs and Reagents

Glucosamine (Glu) and cyclosporine A (CsA) were purchased from Sigma-Aldrich Co. (St. Louis, Mo., USA) and Chong Kun Dang Pharm. (Seoul, Korea), respectively. In addition, IMQ cream (5%) (Aldara) was purchased from Dong-A Pharm.

### 1-3. Induction of Psoriasis-Like Lesions in C57BL/6 Mice

IMQ cream (5%) (Aldara; 3M Health Care, UK) was used to induce psoriasis-like skin symptoms in C57BL/6 mice.

More specifically, hair on the backs of C57BL/6 mice was shaved off using an electric razor and then treated with a depilatory (Niclean, Ildong, Korea). For all other groups except a normal group, 62.5 mg of IMQ cream was applied daily to the shaved back area for 14 days.

On the seventh day after the first sensitization, mice were divided into several groups, and glucosamine and cyclosporine A were administered at various doses, individually or in combination. On the other hand, the normal group as a negative control group was treated only with a vehicle cream (petrolatum).

### 1-4. Calculation of Skin Lesion Score

The degree of (1) erythema, (2) scaling and (3) thickening was scored as 0 (none), 1 (mild), 2 (moderate) and 3 (severe). A cumulative dermatitis score (erythema+scaling+thickening) indicates the severity of psoriasis in back skin (scale: 0-9).

### 1-5. Cytokine Production Assay

To measure cytokine levels in blood serum, mouse blood serum was collected 24 hours after the final administration and stored at −70° C. until analysis. To measure cytokine levels in the skin tissues, the back skin of mice was removed and stored at −80° C. Thereafter, the skin tissues were homogenized using a Bullet Blender™ Blue (Next Advance, Averill Park, N.Y.) at 4° C. to obtain supernatants, and the supernatants were stored at −30° C. IL-17A, IL-22, IL-23, and TNF-α concentrations in the blood serum and the skin tissues of mice were measured using the Mouse IL-17A Quantikine ELISA Kit (R&D Systems, Minneapolis, Minn., USA), IL-22, IL-23 and TNF-α ELISA kits (eBioscience, San Diego, Calif., USA). Meanwhile, ELISA was performed according to the manufacturer's instructions.

### 1-6. Histological Analysis

Mouse back skin which was fixed with paraformaldehyde and paraffin-embedded was sliced and stained with hematoxylin and eosin (H&E). Based on histological findings, several representative symptoms of psoriasis (epidermal thickening, stratum corneum and inflammatory cell infiltration) were assessed in the epidermis or dermis in a blind manner.

### 1-7. Flow Cytometry of Regulatory T (Treg) Cells

Single-cell suspensions prepared from spleens were stained with FITC-anti-CD4 (GK1.5) and PE-anti-CD25 (280406) (R&D Systems, Minneapolis, Minn., USA), and incubated in a PBS solution containing 2% FBS at 4° C. for 30 minutes under dark conditions. After incubation, the samples were washed twice and analyzed using CellQuest software (Becton Dickinson).

### Example 2. Verification of Effect of Single Administration of Glucosamine and Cyclosporine A (CsA) on Psoriasis Treatment

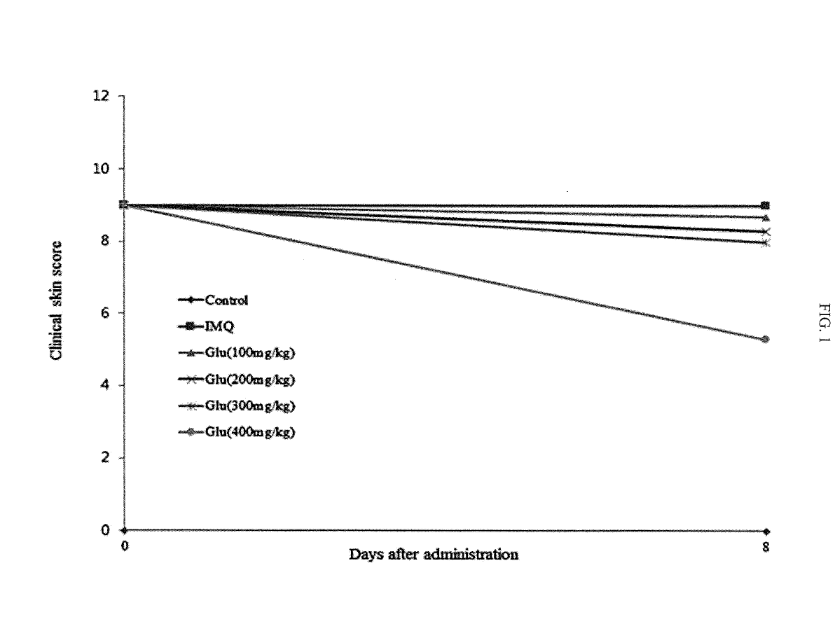

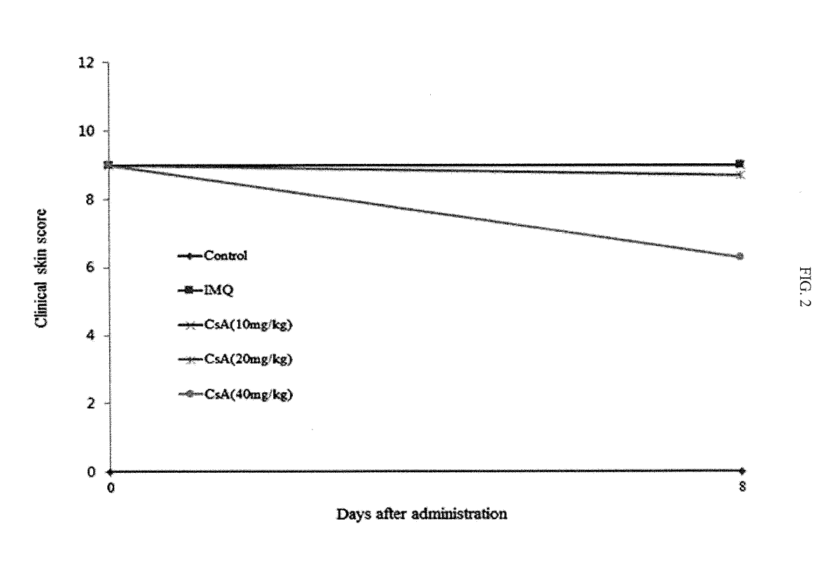

Glucosamine (100, 200, 300 and 400 mg/kg) and cyclosporine A (10, 20, and 40 mg/kg) were orally administered to the psoriasis-like mouse model described in Example 1-3 at different doses for 7 days, respectively. Then, according to the method described in Example 1-4, several representative symptoms were measured using cumulative scores (erythema+scaling+thickening), and the results are shown in FIGS. 1 and 2.

As shown in FIGS. 1 and 2, when high doses of glucosamine (400 mg/kg) and cyclosporine A (40 mg/kg) were administered, respectively, the severity of psoriasis-like skin lesions in mice was reduced. However, when low doses of glucosamine (100, 200 and 300 mg/kg) and cyclosporine A (10 and 20 mg/kg) were administered, respectively, there was no significant effect.

Thus, in the following examples, experiments were conducted with low doses of glucosamine (300 mg/kg) and cyclosporine A (10 and 20 mg/kg).

### Example 3. Verification of Effect of Co-Administration of Low Doses of Glucosamine and Cyclosporine A (CsA) on Psoriasis Treatment

### 3-1. Confirmation of Effect by Comparison of Clinical Skin Scores

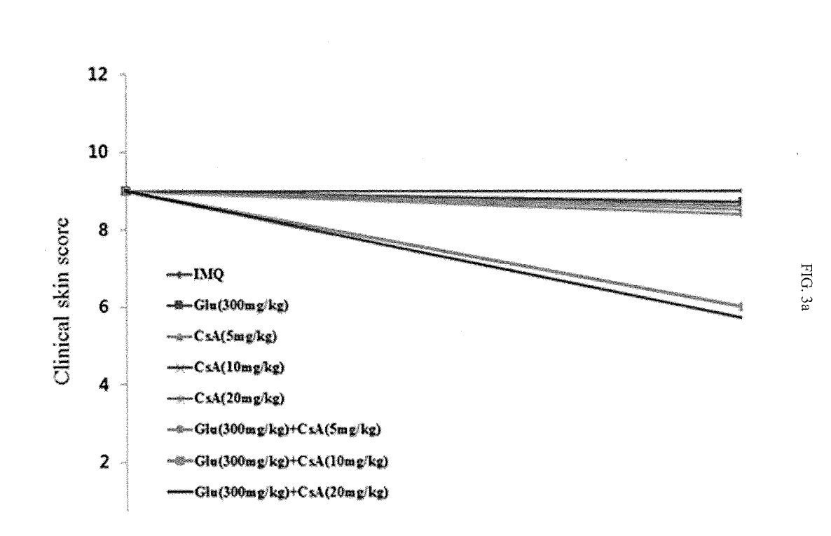

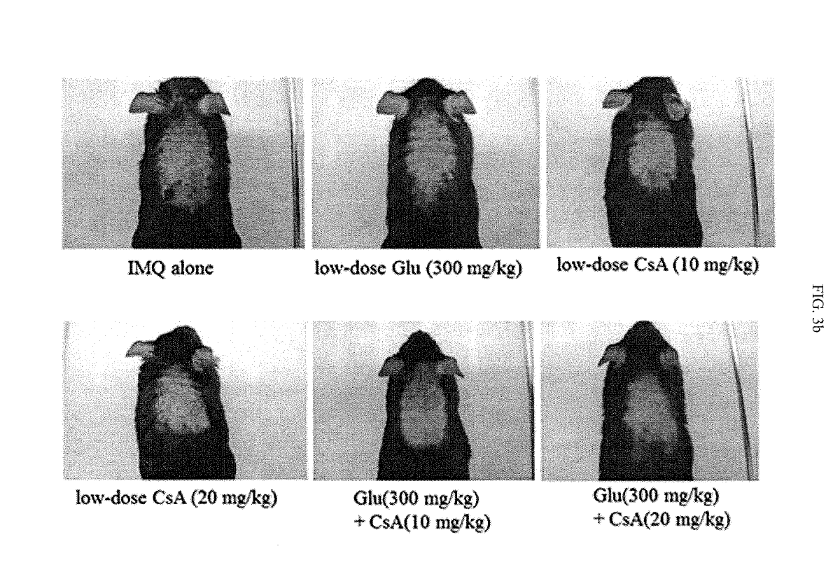

Low doses of glucosamine (300 mg/kg) and cyclosporine A (10 and 20 mg/kg) are orally co-administered to the psoriasis-like mouse model described in Example 1-3 once a day for a week. According to the method described in Example 1-4, clinical skin scores were determined and the results are shown in FIG. 3a. The clinical skin scores were calculated by summing the scores obtained on the basis of symptoms of erythema, scaling and thickening. In addition, the clinical characteristics of mice in each case are shown in FIG. 3b.

As shown in FIGS. 3a and 3b, compared with administration of low doses of glucosamine and cyclosporine A alone, co-administration of low doses of glucosamine (300 mg/kg) and cyclosporine A (10 and 20 mg/kg) improved clinical skin scores and clinical appearance and reduced lesions. On the other hand, there was no significant difference between administration of low doses of glucosamine and cyclosporine A alone and the control group (IMQ alone).

### 3-2. Confirmation of Effect by Comparison of Cytokine Levels

To evaluate the effect of co-administration of glucosamine and cyclosporine A (CsA) on psoriasis treatment, the following experiments were conducted to measure changes in immune responses.

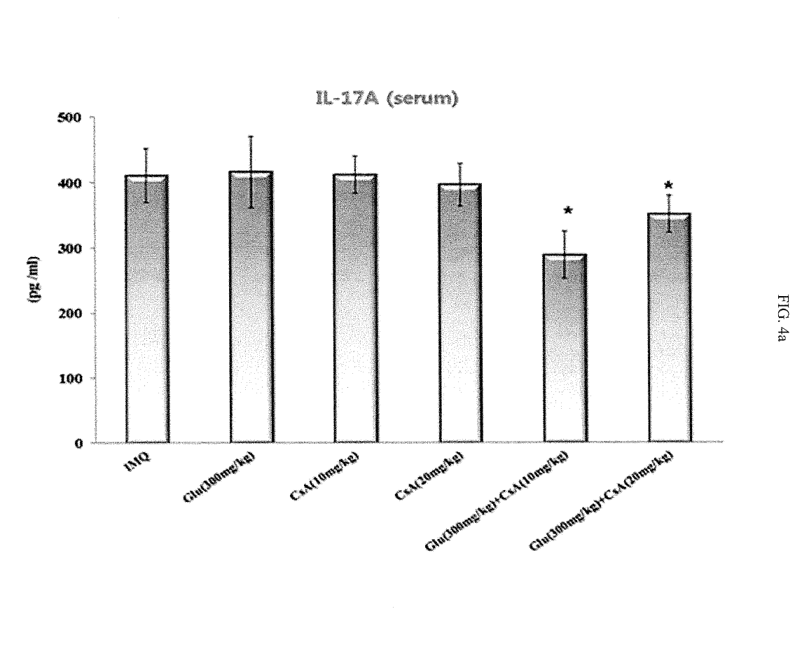

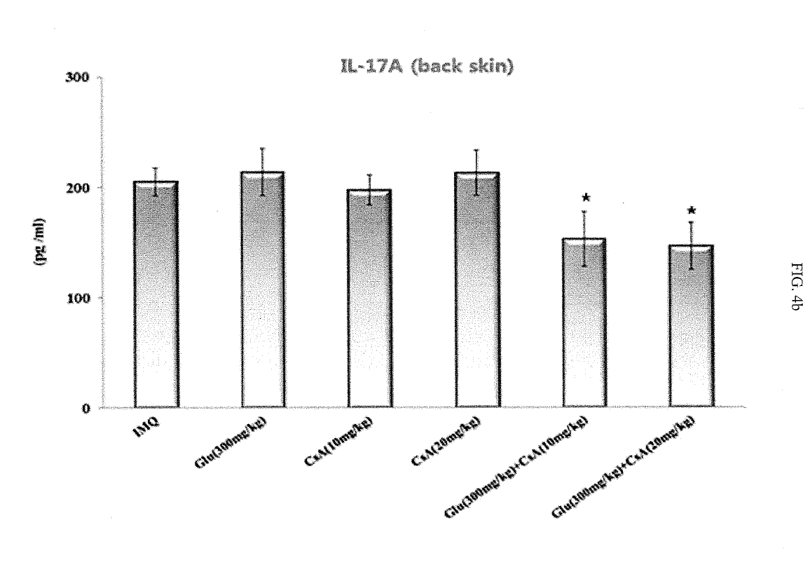

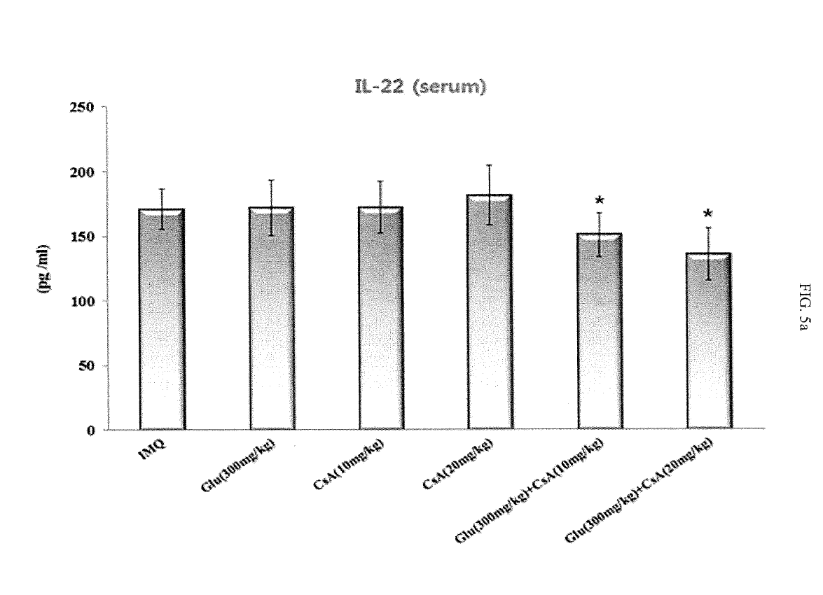

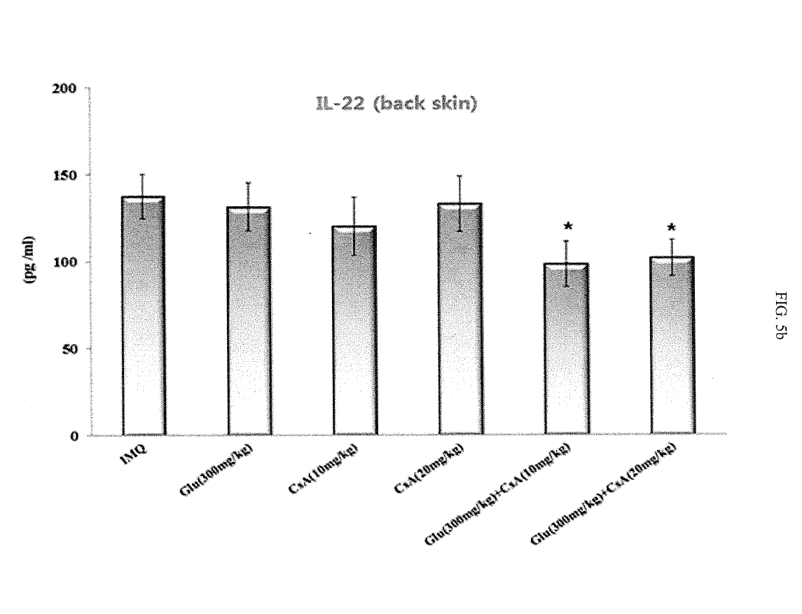

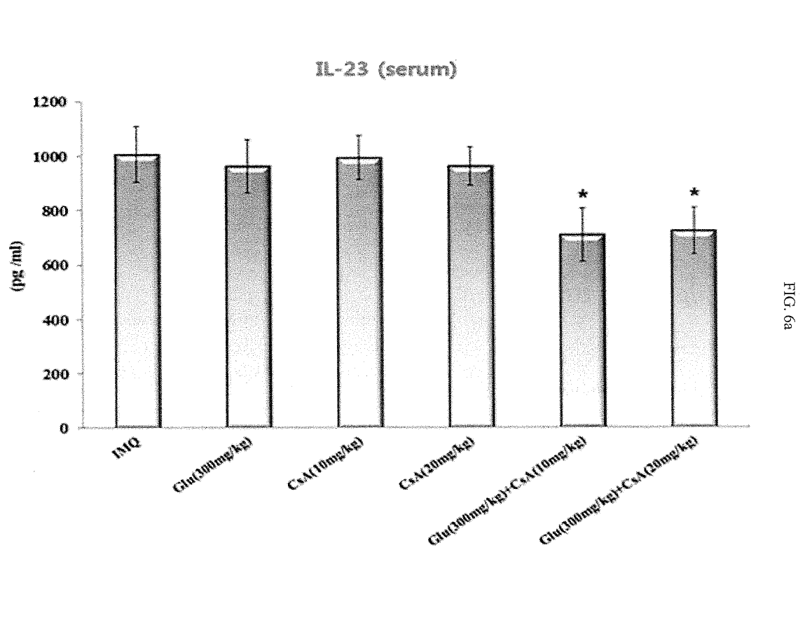

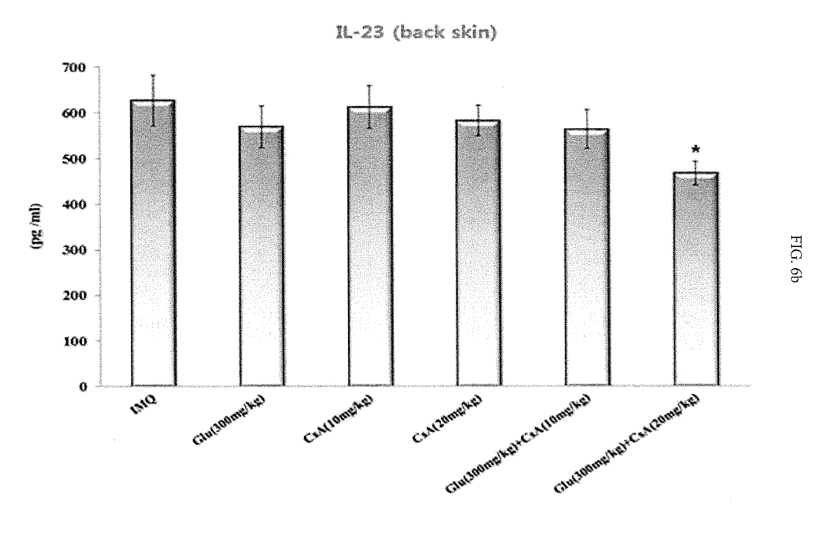

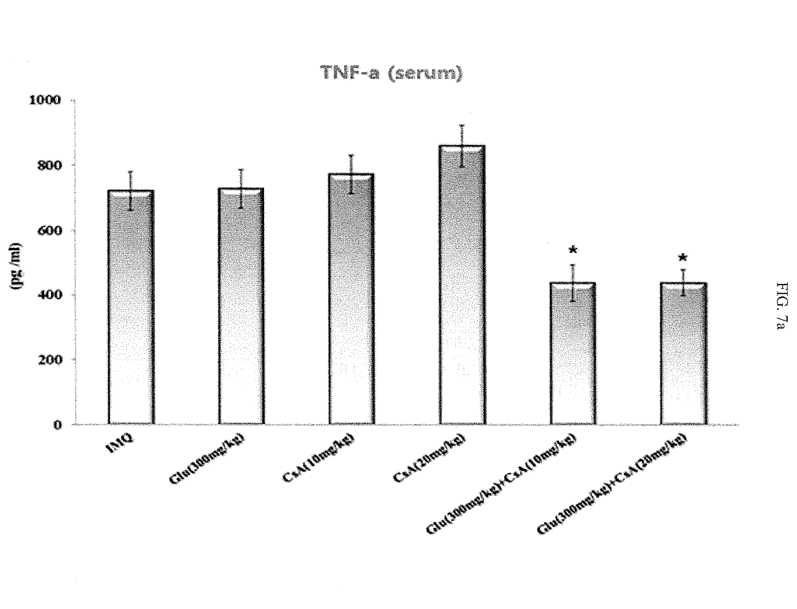

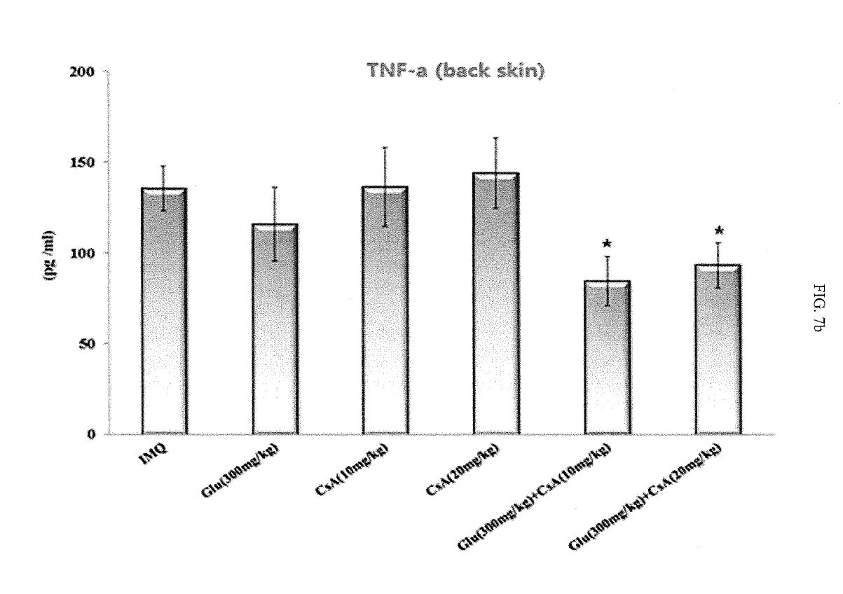

That is, after low doses of glucosamine (300 mg/kg) and cyclosporine A (10 and 20 mg/kg) were administered to the psoriasis-like mouse model described in Example 1-3, alone or in combination, according to the method described in Example 1-5, the levels of cytokines (IL-17A, IL-22, IL-23 and TNF-α) were measured in blood serum samples and skin samples, and the results are shown in FIGS. 4 to 7, respectively.

As shown in FIG. 4 to FIG. 7, compared with administration of low doses of glucosamine and cyclosporine A alone, in both blood serum samples and skin samples, co-administration of low doses of glucosamine (300 mg/kg) and cyclosporine A (10 and 20 mg/kg) significantly reduced the Th17-mediated cytokine levels of IL-17A, IL-22 and IL-23. In addition, co-administration of low doses of glucosamine (300 mg/kg) and cyclosporine A (10 and 20 mg/kg) significantly reduced the level of TNF-α, a Th1-associated pro-inflammatory cytokine. On the other hand, there was no significant difference between administration of low doses of glucosamine and cyclosporine A alone and the control group (IMQ alone).

### 3-3. Confirmation of Effect by Comparison of Histopathological Features

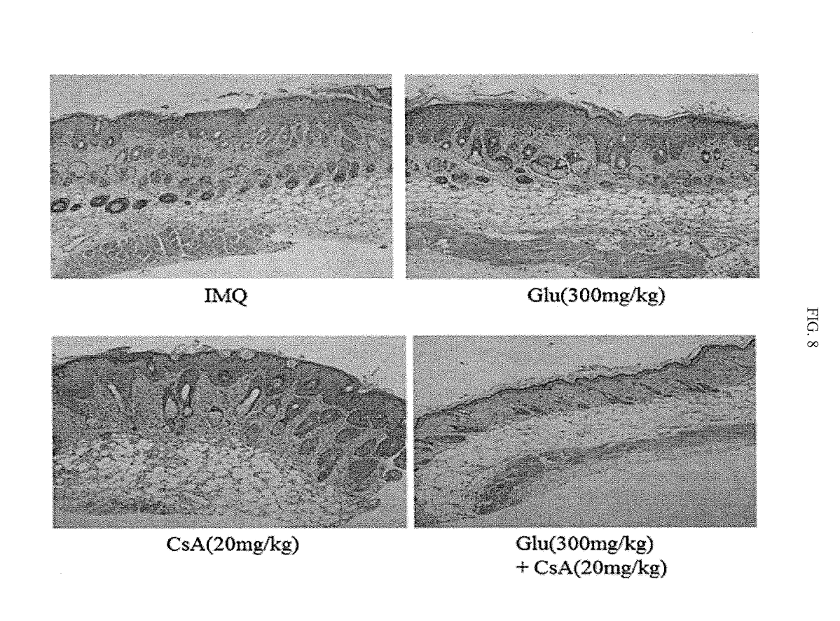

After low doses of glucosamine (300 mg/kg) and cyclosporine A (20 mg/kg) were administered to the psoriasis-like mouse model described in Example 1-3, alone or in combination, according to the method described in Example 1-6, H&E staining for back skin samples was performed, and the results are shown in FIG. 8.

As shown in FIG. 8, histopathological features, such as epidermal thickening, parakeratosis, hyperkeratosis and infiltration of inflammatory cells into the epidermis and dermis, were observed in the back skin of IMQ-induced mice. However, compared with administration of low doses of glucosamine and cyclosporine A alone, co-administration of low doses of glucosamine (300 mg/kg) and cyclosporine A (20 mg/kg) significantly reduced epidermal thickening, parakeratosis and infiltration of cells into the dermis.

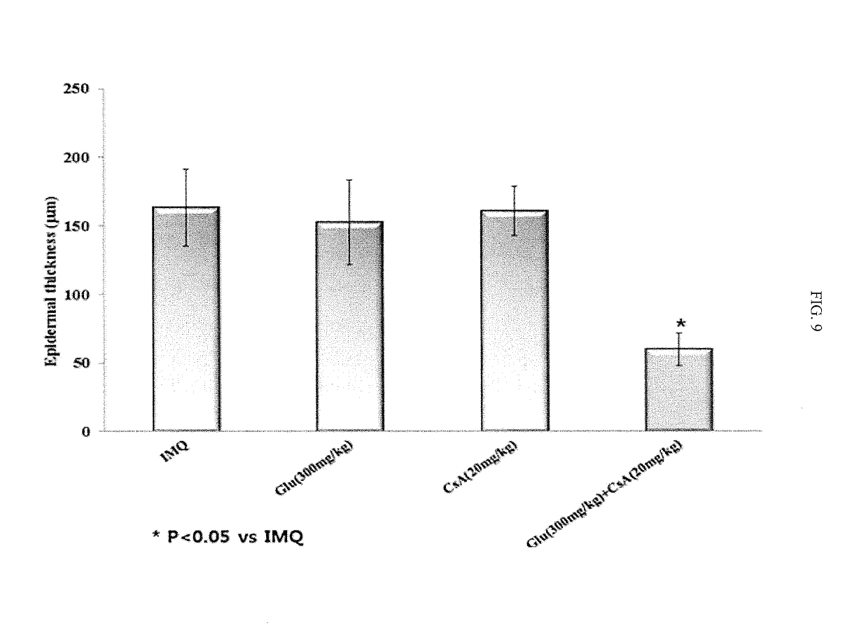

In addition, after low doses of glucosamine (300 mg/kg) and cyclosporine A (20 mg/kg) were administered alone or in combination, the epidermal thicknesses of back skin samples was measured, and the results are shown in FIG. 9.

As shown in FIG. 9, compared with administration of low doses of glucosamine and cyclosporine A alone, co-administration of low doses of glucosamine (300 mg/kg) and cyclosporine A (20 mg/kg) significantly reduced epidermal thickness.

### 3-4. Confirmation of Effect by Comparison of Spleen Weight

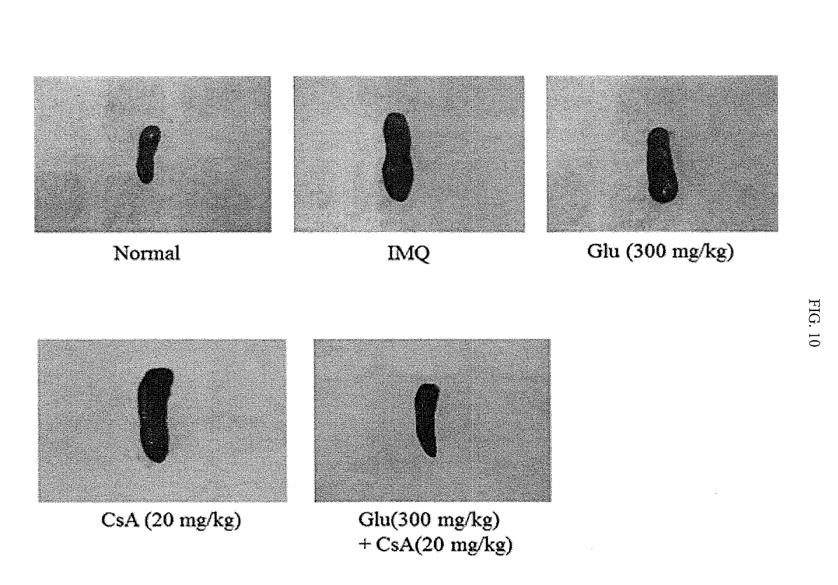

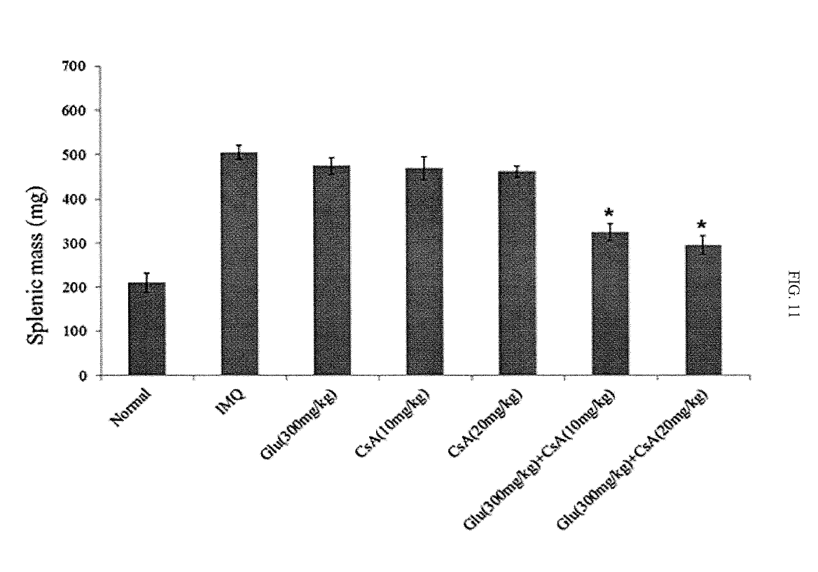

Previous studies reported that the size and weight of the spleen significantly increased in an IMQ-induced psoriasis mouse model. Accordingly, after low doses of glucosamine (300 mg/kg) and cyclosporine A (20 mg/kg) were administered to the psoriasis-like mouse model described in Example 1-3, alone or in combination, mice were sacrificed and spleens were removed to determine the size and weight of each spleen, and the results are shown in FIGS. 10 and 11, respectively.

As shown in FIGS. 10 and 11, compared with administration of low doses of glucosamine and cyclosporine A alone, co-administration of low doses of glucosamine (300 mg/kg) and cyclosporine A (20 mg/kg) significantly reduced spleen size and weight.

### 3-5. Confirmation of Effect by Comparison of Regulatory T (Treg) Cell Number

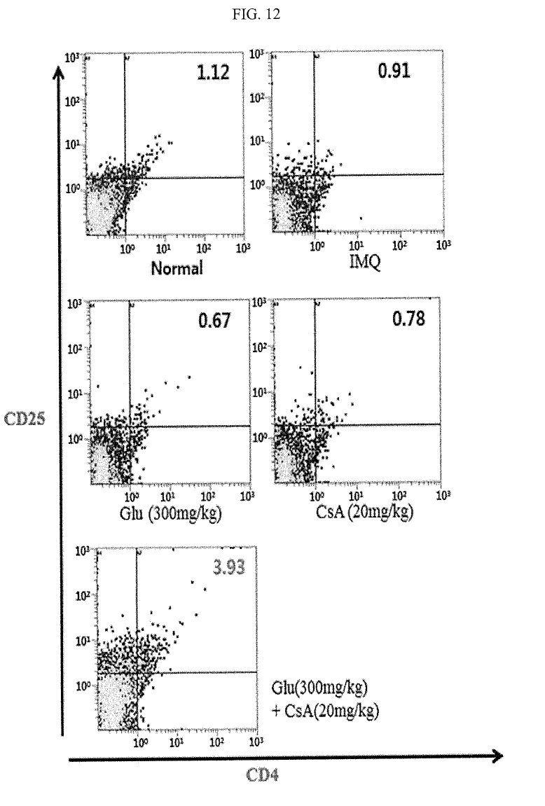

After low doses of glucosamine (300 mg/kg) and cyclosporine A (20 mg/kg) were administered to the psoriasis-like mouse model described in Example 1-3, alone or in combination, according to the method described in Example 1-7, the number of CD4⁺ CD25⁺ Treg cells was measured, and the results are shown in FIG. 12.

As shown in FIG. 12, compared with administration of low doses of glucosamine and cyclosporine A alone, co-administration of low doses of glucosamine (300 mg/kg) and cyclosporine A (20 mg/kg) significantly reduced the number of CD4⁺ CD25⁺ Treg cells. On the other hand, there was no significant difference between administration of low doses of glucosamine and cyclosporine A alone and the control group (IMQ alone).

The aforementioned description of the present invention is provided by way of example and those skilled in the art will understood that the present invention can be easily changed or modified into other specified forms without departing from the technical spirit or essential characteristics of the present invention. Therefore, it should be understood that the aforementioned examples are only provided by way of example and not provided to limit the present invention.

1. A pharmaceutical composition for preventing or treating psoriasis comprising cyclosporine and glucosamine as active ingredients.

2. The pharmaceutical composition according to claim 1, wherein the cyclosporine and glucosamine are contained in a weight ratio of 1:5 to 1:2000.

3. The pharmaceutical composition according to claim 1, wherein the cyclosporine is cyclosporine A.

4. A cosmetic composition for improving psoriasis comprising cyclosporine and glucosamine as active ingredients.

5. The cosmetic composition according to claim 4, wherein the cyclosporine and glucosamine are contained in a weight ratio of 1:5 to 1:2000.

6. The cosmetic composition according to claim 4, wherein the cyclosporine is cyclosporine A.

7. A pharmaceutical composition for enhancing effectiveness of anti-psoriasis treatment, which is used in combination with an anti-psoriasis agent, wherein the pharmaceutical composition comprises glucosamine and the anti-psoriasis agent is cyclosporine.

8. The pharmaceutical composition according to claim 7, wherein the pharmaceutical composition and the anti-psoriasis agent are administered simultaneously, separately or sequentially.

9. A method of treating psoriasis, the method comprising co-administering cyclosporine and glucosamine to an individual.

10. A method of enhancing effectiveness of anti-psoriasis treatment, the method comprising co-administering an anti-psoriasis agent and glucosamine to an individual, wherein the anti-psoriasis agent is cyclosporine.
